(19) United States
(12) Patent Application Publication
(10) Pub. No.: US 2025/0261904 A1
(43) Pub. Date: Aug. 21, 2025

(54) CIRCADIAN RHYTHM ANALYSIS DEVICE AND OPERATION METHOD THEREOF

(71) Applicant: KOOKMIN UNIVERSITY INDUSTRY ACADEMY COOPERATION FOUNDATION, Seoul (KR)

(72) Inventors: Dae hwan KIM, Seoul (KR); Chang wook KIM, Seoul (KR); Young Rag DO, Seoul (KR); Sun woong CHOI, Seoul (KR); Seung Min LEE, Hanam-si Gyeonggi-do (KR); Yun Jae EO, Seoul (KR); Seo Hyeon KIM, Goyang-si Gyeonggi-do (KR); Jin Gyu PARK, Goyang-si Gyeonggi-do (KR); Dong Uk KIM, Suwon-si Gyeonggi-do (KR)

(21) Appl. No.: 19/200,779

(22) Filed: May 7, 2025

Related U.S. Application Data
(63) Continuation of application No. PCT/KR2023/017608, filed on Nov. 6, 2023.
(63) Continuation of application No. PCT/KR2023/095073, filed on Nov. 15, 2023.

(30) Foreign Application Priority Data
Nov. 7, 2022 (KR) 10-2022-0147216
Jan. 10, 2023 (KR) 10-2023-0003340

(51) Int. Cl. A61B 5/00; G01J 1/42; G01J 1/44

(52) U.S. Cl. CPC A61B 5/4857; A61B 5/4809; G01J 1/4204; G01J 1/44

(57) ABSTRACT

A device and a method for analyzing a circadian rhythm. The device may include: a transceiver configured to receive a circadian illuminance value; and at least one processor configured to calculate a first circadian rhythm index corresponding to an indicator indicating a shift degree of a phase of a circadian rhythm based on the circadian illuminance value, and calculate a second circadian rhythm index corresponding to an indicator indicating a melatonin secretion suppression degree based on the circadian illuminance value. The first circadian rhythm index and the second circadian rhythm index are indicators indicating an influence of the circadian illuminance on the circadian rhythm, and for example, the first circadian rhythm index or the second circadian rhythm index is calculated by an integral equation using a weight at a specific timing and the circadian illuminance value at a specific timing.

### TECHNICAL FIELD

The technical idea of the present disclosure relates to a circadian rhythm analysis device and an operation method thereof, and particularly, to a circadian rhythm analysis device including a transceiver, and a processor that monitors and quantitatively analyzes changes in a circadian rhythm, and an operation method thereof.

### BACKGROUND ART

A change regulated by a bio-clock existing in a human body, and repeated in a 24-hour cycle is called circadian rhythm (also called “biorhythm” or “circadian rhythm”). This circadian rhythm is most affected by light. That is, humans who have survived in a 24-hour day and night light environment have a resulting circadian rhythm.

If humans are exposed only to natural light that matches a 24-hour time zone, the circadian rhythm would follow a normal circadian rhythm. However, if humans are exposed to artificial light at night, the secretion of melatonin can be suppressed, causing the circadian rhythm to deviate from the normal circadian rhythm and disrupt deep sleep.

That is, according to recent bio-and medical-related research groups, it is reported that light emitted by artificial lighting has a significant impact on human circadian rhythm. In particular, the circadian rhythm is most affected by blue light with a wavelength of about 450 nm to about 470 nm, because blue light of that wavelength suppresses melatonin secretion in humans.

Here, melatonin is a biological compound with antioxidant and anticancer properties, and when the amount of melatonin secreted increases, a human biological clock determines a current time as night, and conversely, when the amount of melatonin secreted decreases, the human biological clock determines the current time as day.

Health-related problems that individuals may experience due to disruption of circadian rhythms include, for example, seasonal affective disorder, sleep disorders, depression, jet lag, and health conditions associated with shift work. To treat these diseases, the circadian rhythm must be properly balanced by suppressing melatonin secretion at an appropriate morning time and facilitating melatonin secretion at an appropriate evening time.

Meanwhile, a general illuminance measurement device measures the illuminance (Lux) of external light using a visual wavelength filter (Visual Lambda Filter; V-λ Filter) that follows the light sensitivity characteristic curve for the human eye, i.e. the visual sensitivity curve. Here, illuminance (Lux) refers to the intensity of light that can be perceived by the human eye, and is also referred to as visual illuminance (Lux) to be distinguished from circadian illuminance (also referred to as “bio-illuminance” or “non-visual illuminance”).

According to this visual sensitivity curve, a maximum sensitivity is achieved in light having a wavelength of approximately 380 nm to 780 nm. However, visual illuminance measured by general illuminance measurement devices that follow the visual sensitivity curve may differ from the circadian illuminance that affects human circadian rhythms. That is, circadian illuminance can be referred to as an intensity of light (i.e., the number of photons) in a wavelength range that affects the circadian rhythm among light components.

General illuminometers do not provide quantitative information on how much external light affects circadian rhythms, but rather measure light intensity relative to the visual illuminance.

In contrast, if information related to the circadian rhythm of users who are exposed to different light environments every day is provided individually, the information can contribute to improving the health of the users. Therefore, there is a need for research on technologies that can monitor, predict, and analyze the circadian rhythm in a user-customized manner.

The above-described content merely provides background information for the present invention and does not correspond to previously disclosed technology.

### SUMMARY

### Technical Problem

An object to be achieved by the technical idea of the present disclosure is to provide a technology for monitoring and analyzing changes in a user's circadian rhythm in real time by using a circadian illuminance value measured by detecting light in a wavelength that affects the circadian rhythm among light components.

Further, an object to be achieved by the technical idea of the present disclosure is to provide a technology for calculating and analyzing circadian rhythm indices corresponding to an indicator indicating a degree of shift in a phase of the circadian rhythm and an indicator indicating a degree of suppression of melatonin secretion based on a circadian illuminance value.

### Technical Solution

In order to achieve the object, a circadian rhythm analysis device according to an aspect of the technical idea of the present disclosure may include: a transceiver configured to receive a circadian illuminance value; and at least one processor configured to calculate a first circadian rhythm index corresponding to an indicator indicating a shift degree of a phase of a circadian rhythm based on the circadian illuminance value, and calculate a second circadian rhythm index corresponding to an indicator indicating a melatonin secretion suppression degree based on the circadian illuminance value. The first circadian rhythm index and the second circadian rhythm index are indicators indicating an influence of the circadian illuminance on the circadian rhythm. Further, the first circadian rhythm index and the second circadian rhythm index are calculated by an integral equation using a weight at a specific timing and the circadian illuminance value at a specific timing.

An operation method for analyzing a circadian rhythm index performed by a circadian rhythm index analysis device according to an aspect of the technical idea of the present disclosure may include: receiving a circadian illuminance value; calculating a first circadian rhythm index corresponding to an indicator indicating a degree of shift in a phase of a circadian rhythm based on the circadian illuminance value; and calculating a second circadian rhythm index corresponding to an indicator indicating a melatonin secretion suppression degree based on the circadian illuminance value. The first circadian rhythm index and the second circadian rhythm index are indicators indicating an influence of the circadian illuminance on the circadian rhythm, and the first circadian rhythm index and the second circadian rhythm index are calculated by an integral equation using a weight at a specific timing and the circadian illuminance value at a specific timing.

### Advantageous Effects

According to a circadian rhythm analysis device and an operation method thereof of the technical idea of the present disclosure, there is an effect in which it is possible to quantitatively predict and analyze changes in the circadian rhythm, which affect a user in a 24-hour cycle, by using the circadian illuminance value detected and measured by the circadian illuminance measurement device.

According to a circadian rhythm analysis device and an operation method thereof of the technical idea of the present disclosure, there is an effect in which by calculating an indicator indicating a degree of shift of a phase of the circadian rhythm and an index representing a degree of suppression of melatonin secretion based on the circadian illuminance value, it is possible to quantitatively calculate an indicator indicating an influence of circadian illuminance on the circadian rhythm.

In addition, according to the circadian rhythm analysis device and the operation method thereof of the technical idea of the present disclosure, there is an effect of being able to provide an indicator indicating the influence of the circadian illuminance on the circadian rhythm by converting the index into a sleep disturbance index or light pollution index that the general public easily understands.

In addition, according to the circadian rhythm analysis device and the operation method thereof of the technical idea of the present disclosure, there is an effect in which it is possible to monitor and analyze different circadian rhythms of users in real time in a 24-hour cycle to provide various indicators of circadian rhythm, thereby usefully guiding individual healthcare for each scenario.

Advantages which can be obtained in the present disclosure are not limited to the aforementioned advantages and other unmentioned advantages will be clearly understood by those skilled in the art from the following description.

### BRIEF DESCRIPTION OF DRAWINGS

FIG. 1 is a block diagram illustrating an electronic device 101 in a network environment according to various embodiments of the present disclosure.

FIG. 2 is a block diagram schematically illustrating a circadian rhythm analysis system 200 according to an embodiment of the present disclosure.

FIG. 3 is a graph showing a circadian rhythm sensitivity curve and a visual sensitivity curve.

FIG. 4 is a schematic block diagram of a circadian illuminance measurement device 400.

FIG. 5 is a schematic block diagram of a circadian rhythm analysis device 500.

FIG. 6 is a conceptual diagram of a model utilized in a first algorithm and a second algorithm according to an embodiment of the present disclosure.

FIG. 7 is a graph showing a phase shift value with respect to a circadian time according to an embodiment of the present disclosure.

FIG. 8 is a graph showing a phase shift value with respect to a local time according to an embodiment of the present disclosure.

FIG. 9 is a graph showing a phase shift value with respect to light irradiation duration according to an embodiment of the present disclosure.

FIG. 10 is a graph showing a phase shift value with respect to a circadian illuminance according to an embodiment of the present disclosure.

FIG. 11 is a graph showing a normalized melatonin value with respect to the local time according to an embodiment of the present disclosure.

FIG. 12 is a graph showing a melatonin suppression value with respect to the light irradiation duration according to an embodiment of the present disclosure.

FIG. 13 is a graph showing the melatonin suppression value with respect to the circadian illuminance according to an embodiment of the present disclosure.

FIG. 14 is a graph showing lens transmittance according to a wavelength by each user's age according to an embodiment of the present disclosure.

FIG. 15 is a graph showing a ratio of lens transmittance according to a wavelength compared to a 32-year-old user according to an embodiment of the present disclosure.

FIG. 16 is a flowchart showing a method for analyzing a circadian rhythm index of a circadian rhythm analysis device according to an embodiment of the present disclosure.

### DETAILED DESCRIPTIONS OF EXEMPLARY EMBODIMENTS

Hereinafter, embodiments of the present disclosure will be described in detail with reference to the accompanying drawings.

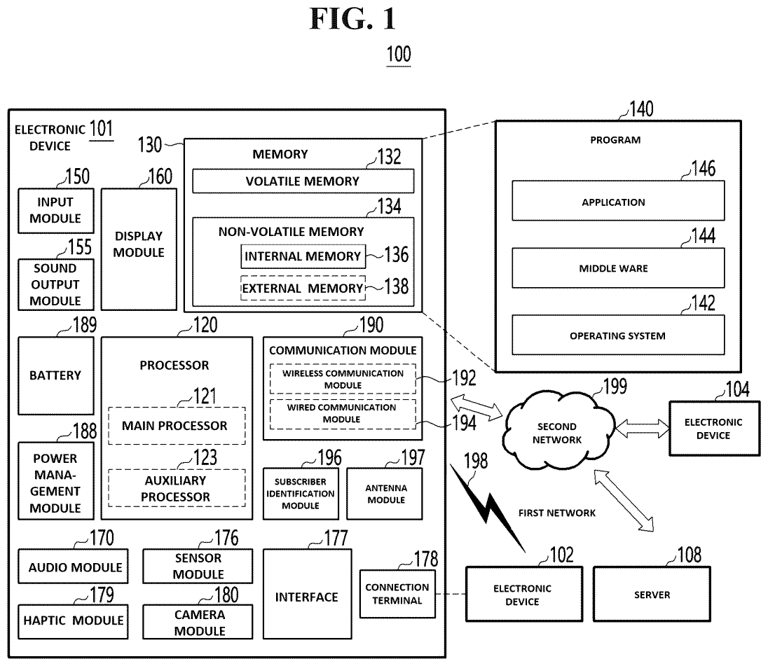

FIG. 1 is a block diagram of an electronic device 101 in a network environment according to various embodiments of the present disclosure.

Referring to FIG. 1, in the network environment 100, the electronic device 101 may communicate with an electronic device 102 through a first network 198 (e.g., a short-range wireless communication network) or communicate with an electronic device 104 or a server 108 through a second network 199 (e.g., a long-range wireless communication network). According to an embodiment, the electronic device 101 may communicate with the electronic device 104 through the server 108. According to an embodiment, the electronic device 101 may include a processor 120, a memory 130, an input module 150, a sound output module 155, a display module 160, an audio module 170, a sensor module 176, an interface 177, a connection terminal 178, a haptic module 179, a camera module 180, a power management module 188, a battery 189, a communication module 190, a subscriber identification module 196, or an antenna module 197. In some embodiments, in the electronic device 101, at least one (e.g., the connection terminal 178) of the components may be omitted from or one or more other components may be added. In some embodiments, some (e.g., the sensor module 176, the camera module 180, or the antenna module 197) of the components may be integrated into a single component (e.g., the display module 160).

For example, the processor 120 may execute software (e.g. the program 140) to control at least one other component (e.g., a hardware or software component) of the electronic device 101 connected to the processor 120, and perform various data processing or operations. According to an embodiment, as at least some of the data processing or operations, the processor 120 may store, in a volatile memory 132, instructions or data received from other components (e.g., the sensor module 176 or the communication module 190), process the commands or data stored in the volatile memory 132, and store result data in a non-volatile memory 134. According to an embodiment, the processor 120 may include a main processor 121 (e.g., a central processing unit or an application processor) or an auxiliary processor 123 (e.g., a graphic processing unit, a neural processing unit (NPU), an image signal processor, a sensor hub processor, or a communication processor) which is operable independently from or together with the main processor 121. For example, when the electronic device 101 includes a main processor 121 and an auxiliary processor 123, the auxiliary processor 123 may be configured to use lower power than the main processor 121 or to be specialized for a given function. The auxiliary processor 123 may be implemented separately from the main processor 121 or as a part of the main processor 121.

For example, the auxiliary processor 123 may control at least some of the functions or states related to at least one component (e.g., the display module 160, the sensor module 176, or the communication module 190) of the components of the electronic device 101 instead of the main processor 121 while the main processor 121 is in an inactive (e.g., sleep or idle) state or together with the main processor 121 while the main processor 121 is in an active (e.g., application execution) state. According to an embodiment, the auxiliary processor 123 (e.g., an image signal processor or a communication processor) may be implemented as a part of another component (e.g., the camera module 180 or the communication module 190) which is functionally related. According to an embodiment, the auxiliary processor 123 (e.g., a neural network processing unit) may include a hardware structure specialized for processing an artificial intelligence model. The artificial intelligence model may be generated through machine learning. The learning may be performed by the electronic device 101 itself in which artificial intelligence is performed, and also performed through a separate server (e.g., a server 108). A learning algorithm may include, for example, supervised learning, unsupervised learning, semi-supervised learning, or reinforcement learning, but is not limited to the above-described example. The artificial intelligence model may include a plurality of artificial neural network layers. The artificial neural network may be one of a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-networks, or a combination of the two or more, but is not limited to the above-described example. In addition to the hardware structure, the artificial intelligence model may additionally or alternatively include a software structure.

The memory 130 may store various data used by at least one component (e.g., the processor 120 or the sensor module 176) of the electronic device 101. The data may include, for example, software (e.g., the program 140) and input data or output data for a command related to the software. The memory 130 may include the volatile memory 132 or the non-volatile memory 134.

The program 140 may be stored in the memory 130 as the software, and may include, for example, an operating system 142, middleware 144, or an application 146.

The input module 150 may receive the command or data to be used for the component (e.g., the processor 120) of the electronic device 101 from the outside (e.g., a user) of the electronic device 101. The input module 150 may include, for example, a microphone, a mouse, a keyboard (e.g., a button), or a digital pen (e.g., a stylus pen).

The input module 150 may receive the command or data to be used for the component (e.g., the processor 120) of the electronic device 101 from the outside (e.g., a user) of the electronic device 101. The input module 150 may include, for example, a microphone, a mouse, a keyboard (e.g., a button), or a digital pen (e.g., a stylus pen).

The sound output module 155 may output a sound signal to the outside of the electronic device 101. The sound output module 155 may include, for example, a speaker or a receiver. The speakers may be used for general purposes such as playing multimedia or playing recordings. The receiver may be used to receive incoming calls. According to an embodiment, the receiver may be implemented separately from the speaker or as a part of the speaker.

The display module 160 may visually provide information to the outside (e.g., the user) of the electronic device 101. The display module 160 may include, for example, a display, a hologram device, or a projector and a control circuit for controlling the corresponding device. According to one embodiment, the display module 160 may include a touch sensor configured to detect a touch, or a pressure sensor configured to measure the intensity of a force generated by the touch.

The audio module 170 may convert a sound into an electric signal, and reversely, convert the electric signal into the sound. According to an embodiment, the audio module 170 may acquire the sound through the input module 150 or output the sound through an external electronic device (e.g., the electronic device 102) (e.g., the speaker or a head phone) directly or wirelessly connected to the sound output module 155 or the electronic device 101.

The sensor module 176 may sense an operation state (e.g., power or temperature) of the electronic device 101 or an external environmental state (e.g., a user state) and generate an electric signal or a data value corresponding to the sensed state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a bio sensor, a temperature sensor, or a humidity sensor. According to an embodiment, the sensor module 176 may include a illuminance sensor for measuring visual illuminance or a circadian illuminance measuring sensor for measuring circadian illuminance.

The interface 177 may support one or more designated protocols which may be used for the electronic device 101 to be directly or wirelessly connected to the external electronic device (e.g., the electronic device 102). According to an embodiment, the interface 177 may include, for example, a high definition multimedia interface (HDMI), a universal serial bus (USB) interface, an SD card interface, an audio interface, or an inter-integrated circuit (I²C) interface.

The connection terminal 178 may include a connector for the electronic device 101 to be physically connected to the external electronic device (e.g. the electronic device 102) through the interface 177. According to an embodiment, the connection terminal 178 may include, for example, an HDMI connector, a USB connector, an SD card connector, or an audio connector (e.g., a head phone connector).

The haptic module 179 may convert the electric signal into a mechanical stimulus (e.g., vibration or motion) or an electrical stimulus which the user may recognize through a tactile or exercise sensation. According to an embodiment, the haptic module 179 may include, for example, a motor, a piezoelectric element, or an electrical stimulus device.

The camera module 180 may photograph a still image and a moving picture. According to an embodiment, the camera module 180 may include one or more lenses, image sensors, image signal processors, or flashes.

The power management module 188 may manage power supplied to the electronic device 101. According to an embodiment, the power management module 188 may be implemented as at least a part of a power management integrated circuit (PMIC), for example.

The battery 189 may provide power to at least one component of the electronic device 101. According to an embodiment, the battery 189 may include, for example, a recharge impossible primary battery, a rechargeable secondary battery, or a fuel cell.

The communication module 190 may support establishment of a direct (e.g., wired) communication channel or a wireless communication channel between the electronic device 101 and the external electronic device (e.g., the electronic device 102, the electronic device 104, or the server 108) and communication execution through the established communication channel. The communication module 190 may include one or more communication processors which are operated independently from the processor 120 (e.g., the application processor) and include one or more communication processors supporting the direct (e.g., wired) communication or the wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module, or a power line communication module). A corresponding communication module among the communication modules may communicate with the external electronic device through a first network 198 (e.g., a short-range communication network such as Bluetooth, wireless fidelity (Wi-Fi) direct or infrared data association (IrDA)) or a second network 199 (e.g., a long-range communication network such as a legacy cellular network, a 5G network, a next-generation communication network, the Internet, or a computer network (e.g., LAN or WLAN)). The types of communication modules may be integrated into one component (e.g., single chip), or may be implemented as a plurality of separate components (e.g., plural chips). The wireless communication module 192 may check or authenticate the electronic device 101 in the communication network such as the first network 198 or the second network 199 by using subscriber information (e.g., international mobile subscriber identity (IMSI)) stored in the subscriber identification module 196.

The wireless communication module 192 may support a 5G network and next-generation communication technology after a 4G network, for example, NR access technology (new radio access technology). The NR access technology may support high-speed transmission of high-capacity data (enhanced mobile broadband (eMBB)), minimizing terminal power and connecting multiple terminals (massive machine type communications (mMTC)), or high reliability and low latency (ultra-reliable and low-latency communications (URLLC)). The wireless communication module 192 may support a high frequency band (e.g., mmWave band) to achieve a high data transmission rate, for example. The wireless communication module 192 may support various technologies for securing performance in the high-frequency band, such as beamforming, massive multiple-input and multiple-output (MIMO), full dimensional MIMO (FD-MIMO), array antenna, analog beam-forming, or large scale antenna. The wireless communication module 192 may support various requirements specified in an electronic device 101, an external electronic device (e.g., an electronic device 104), or a network system (e.g., a second network 199). According to an embodiment, the wireless communication module 192 may support a peak data rate (e.g., 20 Gbps or more) for eMBB realization, a loss coverage (e.g., 164 dB or less) for mMTC realization, or a U-plane latency (e.g., 0.5 ms or less for downlink (DL) and uplink (UL) each, or 1 ms or less for round trip) for URLLC realization.

The antenna module 197 may transmit the signal or power to the outside (e.g., the external electronic device) or receive the signal or power from the outside. According to an embodiment, the antenna module may include an antenna including a radiator formed of a conductor or conductive pattern formed on a substrate (e.g., a PCB). According to one embodiment, the antenna module 197 may include a plurality of antennas (e.g., an array antenna). In such a case, at least one antenna suitable for a communication method used in a communication network, such as the first network 198 or the second network 199, may be selected from the plurality of antennas, for example, by the communication module 190. The signal or power may be transmitted or received between the communication module 190 and the external electronic device through the at least one selected antenna. According to some embodiments, other components (e.g., a radio frequency integrated circuit (RFIC)) may be additionally formed as part of the antenna module 197 in addition to the radiator.

According to various embodiments, the antenna module 197 may form an mmWave antenna module. According to an embodiment, the mmWave antenna module may include a printed circuit board, an RFIC positioned on or adjacent to a first side (e.g., a bottom side) of the printed circuit board and capable of supporting a designated high frequency band (e.g., an mmWave band), and a plurality of antennas (e.g., an array antenna) positioned on or adjacent to a second side (e.g., a top side or a side) of the printed circuit board and capable of transmitting or receiving signals in the designated high frequency band.

At least some of the components may be connected to each other through a communication scheme (e.g., a bus, a general purpose input and output (GPIO), a serial peripheral interface (SPI), or a mobile industry processor interface (MIPI)) between peripheral machines and exchange the signal (e.g., command or data) with each other.

According to an embodiment, the command or data may be transmitted or received between the electronic device 101 and the external electronic device 104 through the server 108 connected to the second network 199. Each of the external electronic device 102 or 104 may be a device of the same type as or a different type from the electronic device 101. According to an embodiment, all or some of the operations executed in the electronic device 101 may be executed in one or more external devices of the external electronic devices 102, 104, or 108. For example, when the electronic device 101 should perform any function or service automatically or in response to a request from the user or another device, the electronic device 101 may request one or more external electronic devices to perform at least a part of the function or service instead of or in addition to autonomously executing the function or service. One or more external electronic devices receiving the request may execute at least a part of the requested function or service or an additional function or service related to the request, and transfer a result of the execution to the electronic device 101. The electronic device 101 may process the result as it is or additionally, and provide the processed result as at least a part of a response to the request. To this end, for example, cloud computing, distributed computing, mobile edge computing (MEC), or client-server computing technology may be used. The electronic device 101 may provide ultra-low latency services, for example, by using distributed computing or mobile edge computing. In another embodiment, the external electronic device 104 may include an Internet of Things (IoT) device. The server 108 may be an intelligent server utilizing machine learning and/or neural networks. According to an embodiment, an external electronic device 104 or server 108 may be included within the second network 199. The electronic device 101 may be applied to intelligent services (e.g., smart home, smart city, smart car, or healthcare) based on 5G communication technology and IoT-related technology.

The electronic devices according to various embodiments disclosed in this document may become various types of devices. The electronic device may include, for example, a portable communication device (e.g., a smartphone), a computer device, a portable multimedia device, a portable medical machine, a camera, a wearable device (e.g., smart glasses), or an appliance device. The electronic device according the embodiment of this specification is not limited to the above-described machines.

Various embodiments of this document and terms used therein are not intended to limit technical features described in this document to specific embodiments, and it should be understood that various embodiments and the terms include various modifications, equivalents, or substitutes for the corresponding embodiment. In connection with the description of the drawings, similar reference numerals may be used for similar or related components. A singular type of a noun corresponding to an item may include one or a plurality of items unless a related context is expressively indicated otherwise. In this document, each of phrases such as “A or B”, “at least one of A and B”, “at least one of A or B”, “A, B, or C”, “at least one of A, B, and C”, and “at least one of A, B, or C” may include at least one or all possible combinations of items listed together with a corresponding phrase among the phrases. Terms such as “first” or “second” may be just used for distinguish a corresponding component from the other corresponding component and the corresponding components are not limited in other aspects (e.g., importance or order). When it is mentioned that any (e.g., first) component is “coupled” or “connected” to the other (e.g., second) component together with a term “functionally” or “communicationally” or without the term, it means that the any component can be connected to the other component directly (e.g., wiredly), wirelessly, or through a third component.

The term “module” used in various embodiments of this document may include a unit implemented as hardware, software, or firmware, and may be used intercompatibly with a term such as logic, a logic block, a part, or a circuit, for example. The module may become an integrally configured part or a minimum unit or a part of the part, which performs one or more functions. For example, according to an embodiment, the module may be implemented in the form of an application-specific integrated circuit (ASIC).

According to various embodiments, each component (e.g., a module or program) of the above-described components may include a single or a plurality of entities, and some of the plurality of entities may be separately disposed in other components. According to various embodiments, one or more components or operations of the above-described components may be omitted, or one or more other components or operations may be added. Alternatively or additionally, the plurality of components (e.g., the module or program) may be integrated into one component. In this case, the integrated component may perform one or more functions of respective components of the plurality of components in the same or similar manner as performing the function by the corresponding component of the plurality of components before the integration. According to various embodiments, operations performed by modules, programs, or other components are executed sequentially in parallel, repeatedly, or heuristically, or one or more of the above operations may be executed in different orders or omitted, or one or more other operations may be added.

Hereinafter, the term “circadian rhythm (or irradiance)” may be referred to as “circadian rhythm (or illuminance)”, “24-hour rhythm (or illuminance)”, “biorhythm (or illuminance)” or “biorhythm (or illuminance)”.

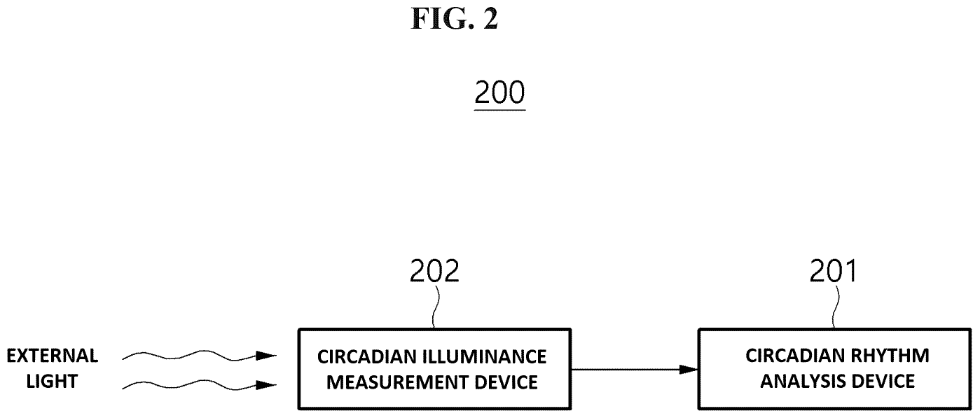

FIG. 2 is a block diagram schematically illustrating a circadian rhythm analysis system 200 according to an embodiment of the present disclosure.

A circadian rhythm analysis system 200 according to an embodiment of the present disclosure as a system for deriving a circadian rhythm index from an external light environment exposed to a user may include a circadian illuminance measurement device 202 and a circadian rhythm analysis device 201. In another embodiment, the circadian illuminance measurement device 202 and the circadian rhythm analysis device 201 may be integrated into one component (e.g., a circadian illuminance measurement and circadian rhythm analysis device).

Referring to FIGS. 1 and 2, the circadian illuminance measurement device 202 in FIG. 2 may be applied to the electronic device 102 (in FIG. 1) of FIG. 1, and the circadian rhythm analysis device 201 in FIG. 2) may be applied to the electronic device 101 (in FIG. 1) of FIG. 1. According to an embodiment, the circadian rhythm analysis device 201 in FIG. 2 may communicate with the circadian illuminance measurement device 202 in FIG. 2 via the first network 198 in FIG. 1.

In an embodiment, the circadian illuminance measurement device 202 may detect external light. The circadian illuminance measurement device 202 may measure circadian illuminance.

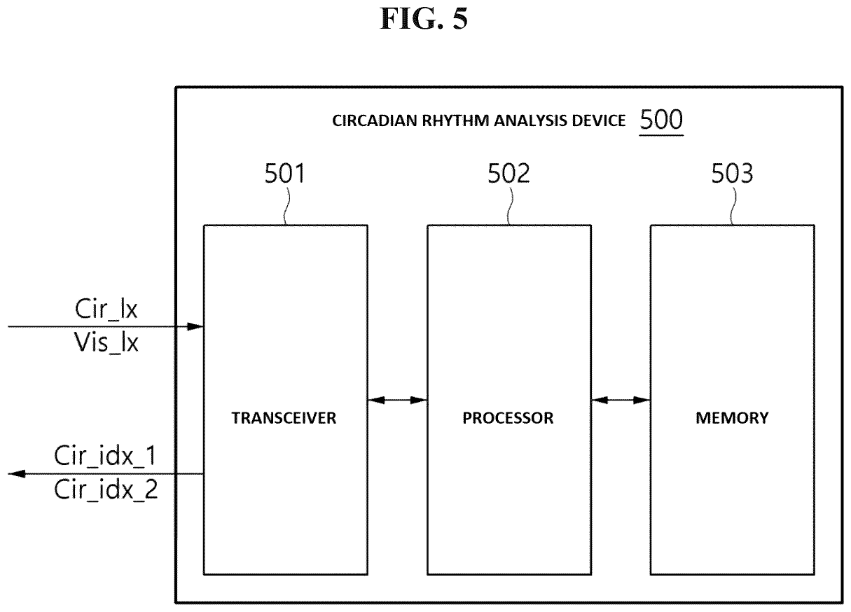

In an embodiment, the circadian rhythm analysis device 201 may receive a circadian illuminance value from the circadian illuminance measurement device 202. The circadian rhythm analysis device 201 may calculate a circadian phase shift (CPS), which corresponds to an indicator indicating a degree of shift in a phase of the circadian rhythm, based on the received circadian illuminance. The circadian rhythm analysis device 201 may calculate a melatonin Suppression Response (MSR), which corresponds to an indicator indicating a degree of melatonin secretion suppression, based on the received circadian illuminance value. The CPS may be referred to as a first circadian rhythm index, and the MSR may be referred to as a second circadian rhythm index. The first circadian rhythm index and the second circadian rhythm index correspond to indicators indicating an influence of the circadian illuminance on the circadian rhythm. Detailed contents on the CPS and the MSR are described below in FIG. 5 and below.

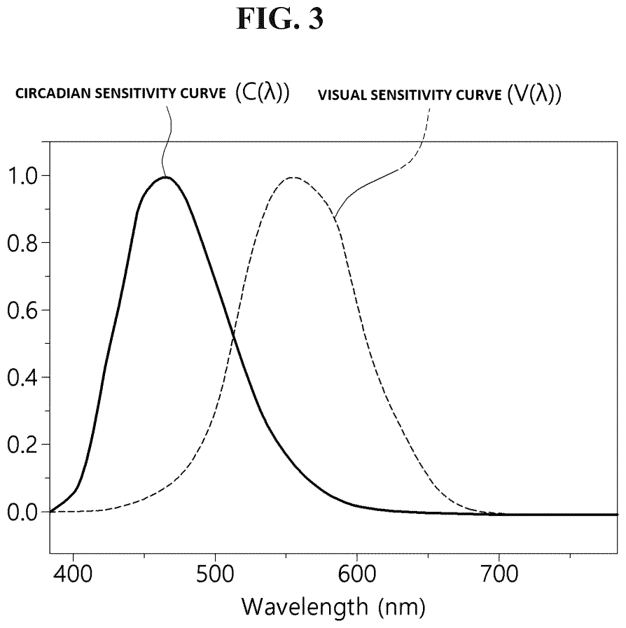

FIG. 3 is a graph showing a circadian sensitivity curve and a visual sensitivity curve.

A circadian sensitivity curve C(λ) as a light sensitivity characteristic curve for hormones that control a human circadian rhythm (e.g., melatonin or cortisol) may be defined as a curve that has a maximum sensitivity in a circadian wavelength band. As an example, the circadian wavelength band may be 400 nm to 600 nm. According to the circadian sensitivity curve, a maximum sensitivity may be achieved in light having the circadian wavelength band.

For example, a circadian wavelength filter following the circadian sensitivity curve may operate as a bandpass filter that passes external light having a wavelength within the circadian wavelength band and blocks external light having a wavelength outside the circadian wavelength band. The circadian wavelength filter transmits light components having the circadian wavelength band, and may have a maximum transmittance at a wavelength of, for example, 490 nm in the circadian wavelength band.

A visual sensitivity curve V(λ) as a light sensitivity characteristic curve for a human eye may be defined as a curve that has a maximum sensitivity in a visual wavelength band. As an example, the visual wavelength band may be 380 nm to 780 nm. According to the visual sensitivity curve, a maximum sensitivity may be achieved in light having the visual wavelength band.

For example, a visual wavelength filter following the visual sensitivity curve may operate as a bandpass filter that passes external light having a wavelength within the visual wavelength band and blocks external light having a wavelength outside the visual wavelength band.

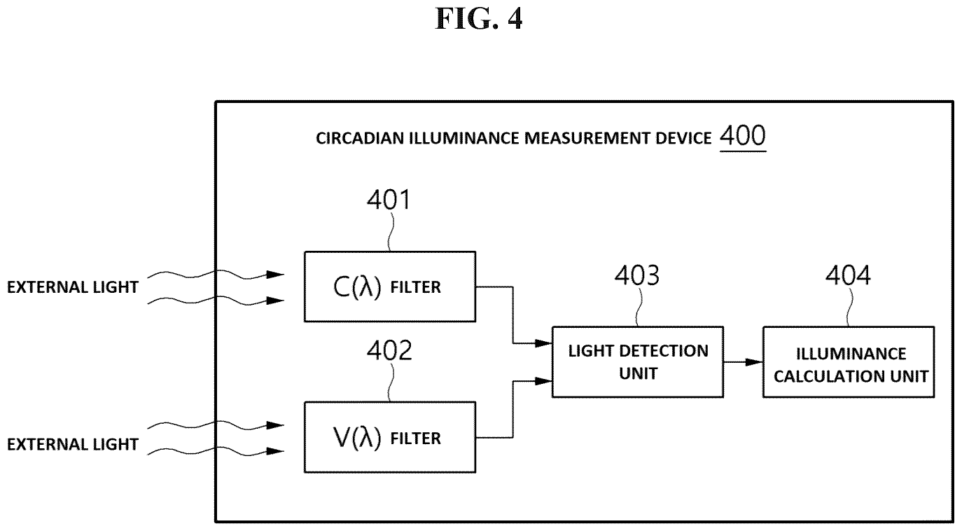

FIG. 4 is a schematic block diagram of a circadian luminance measurement device 400.

Referring to FIGS. 2 and 4, a circadian illuminance measurement device 400 in FIG. 4 may be applied to the circadian illuminance measurement device 202 in FIG. 2.

As an embodiment, the circadian illuminance measurement device 400 may include a C(λ) (or, circadian wavelength) filter (circadian lambda filter; C(λ) Filter) 401 that passes or blocks external light according to the circadian rhythm sensitivity curve, and a V(λ) (or, visual wavelength) filter (visual lambda filter; V(λ) filter) 402 that passes or blocks external light according to the visual sensitivity curve. The circadian illuminance measurement device 400 may include a light detection unit 403 that detects external light passing through the circadian wavelength filter 401 and converts the corresponding light into a circadian wavelength signal and detects external light passing through the visual wavelength filter 402 and converts the corresponding light into a visual wavelength signal, and an illuminance calculation unit 404 that calculates a ratio of the circadian wavelength signal and the visual wavelength signal, calculates a circadian action factor by applying the ratio of the circadian wavelength signal and the visual wavelength signal to a circadian action function that varies according to the visual wavelength signal, and calculates a bio-illuminance value of the external light based on the circadian action factor. The circadian action factor (CAF) as a function of a color temperature of external light may be defined as a ratio of a circadian efficacy of radiation (CER) to a luminous efficacy of radiation (LER). The CAF may be defined as a value which is proportional to the ratio of the circadian wavelength signal and the visual wavelength signal.

As another embodiment, the circadian illuminance measurement device 400 may include a C(λ) filter 401 that passes external light according to the circadian rhythm sensitivity curve, a light detection unit 403 that detects external light passing through the circadian wavelength filter, and converts the detected light into an analog signal (e.g., a voltage value) and outputs the analog signal, and a circadian illuminance calculation unit 404 that converts the analog signal into a digital signal and calculates a circadian illuminance of the external light. The circadian illuminance measurement device 202 may measure the circadian illuminance by passing light having a wavelength of, for example, approximately 450 nm to 550 nm, that is, a circadian wavelength band in which the light sensitivity characteristic for a hormone that controls the circadian rhythm is at a maximum sensitivity, and blocking light having a wavelength other than the wavelength of approximately 450 nm to 550 nm.

For a cool white LED lighting and warm white LED lighting that are measured as the same 500 lx by a general visual illuminance measurement device (e.g., a light meter), different circadian illuminance values may be measured when measured by the circadian illuminance measurement device 400 according to an embodiment of the present disclosure. As an example, the cool white LED lighting with a visual illuminance value of 500 lx may be measured by the circadian illuminance measurement device 400 to have a CAF measured as 0.77 and a circadian illuminance measured as 385 biolux. As an example, the warm white LED lighting with a visual illuminance value of 500 lx may be measured by the circadian illuminance measurement device 400 to have a CAF measured as 0.37 and a circadian illuminance measured as 185 biolux. The biolux may be defined as a unit of circadian illuminance value that may be used in the circadian rhythm measurement device according to an embodiment of the present disclosure.

In addition, the method for measuring the circadian illuminance may be used in various methods, and the above-described examples of the method for measuring the circadian illuminance are merely embodiments, and technical contents of the present disclosure are not limited thereto.

In order to more accurately measure the circadian illuminance in the external light environment exposed to the user, the circadian illuminance measurement device 400 may be directly employed by the user or installed around the user. That is, the circadian illuminance measurement device 400 may be implemented as a wearable device attached to a part of the user's body or installed in a fashion item (e.g., clothing, shoes, hat, bag, hair band, belt, or glasses) worn by the user. In this case, the circadian illuminance measurement device 400 may be manufactured in the form of a clip or badge so that the circadian illuminance measurement device 400 may be easily attached to the fashion item. The circadian illuminance measurement device 400 may be referred to as a bio-illuminometer or a circadian illuminance meter.

FIG. 5 is a schematic block diagram of a circadian rhythm analysis device 500.

Referring to FIGS. 1, 2, and 5, the circadian rhythm analysis device 500 in FIG. 5 may be applied to the electronic device 101 in FIG. 1 or the circadian rhythm analysis device 201 in FIG. 2.

The circadian rhythm analysis device 500 may include a transceiver 501, a processor 502, or a memory 503. This is only an embodiment, and the technical contents of the present disclosure are not limited thereto. In other embodiments, the circadian rhythm analysis device 500 may additionally or alternatively include other components.

The transceiver 501 may receive a circadian illuminance value Cir_lx from the outside. The transceiver 501 may receive a visual illuminance value Vis_lx from the outside. Referring to FIGS. 4 and 5, as an example, the transceiver 501 may receive the circadian illuminance value Cir_lx and/or the visual illuminance value Vis_lx from the circadian illuminance measurement device 400 in FIG. 4.

The transceiver 501 may receive a timing value related to light irradiation from the outside. Referring to FIGS. 4 and 5, as an example, the transceiver 501 may receive the timing value related to the light irradiation from the circadian illuminance measurement device 400 in FIG. 4. As another example, as another example, the transceiver 501 may receive a timing value and/or a duration time value related to light irradiation from a circadian illuminance measurement device 400 in FIG. 4.

The transceiver 501 may transmit a circadian rhythm index Cir to the outside. The circadian rhythm index may be defined as an indicator indicating an influence of the circadian illuminance on the circadian rhythm. The circadian rhythm index Cir may include a first circadian rhythm index Cir_idx_1 and/or a second circadian rhythm index Cir_idx_2. The first circadian rhythm index Cir_idx_1 may be referred to as a circadian phase shift (CPS), which corresponds to an indicator indicating a degree of shift in the phase of the circadian rhythm. The second circadian rhythm index Cir_idx_2 may be referred to as a melatonin suppression response (MSR), which corresponds to an indicator indicating a degree of melatonin secretion suppression.

Referring to FIGS. 1 and 5, as an example, the transceiver 501 may transmit the first circadian rhythm index Cir_idx_1 and/or the second circadian rhythm index Cir_idx_2 to the electronic device 102 in FIG. 1. The first circadian rhythm index Cir_idx_1 may be defined as an indicator indicating the degree of shift in the phase of the circadian rhythm. The second circadian rhythm index Cir_idx_2 may be defined as an indicator indicating the degree of melatonin secretion suppression. The first circadian rhythm index Cir_idx_1 may be referred to as a circadian phase shift (CPS). The second circadian rhythm index Cir_idx_2 may be referred to as a melatonin Suppression Response (MSR).

The processor 502 may control at least one other component of the circadian rhythm analysis device 500. As an example, the processor 502 may control an operation of the transceiver 501 or the memory 503.

The processor 502 may perform processing or calculation of the first circadian rhythm index or the second circadian rhythm index based on the received circadian illuminance value Cir_lx. As an example, the processor 502 may perform the processing or calculation of the first circadian rhythm index Cir_idx_1 or the second circadian rhythm index Cir_idx_2 based on the received circadian illuminance value Cir_lx.

As an embodiment, the processor 502 may calculate the first circadian rhythm index Cir_idx_1 using a first algorithm according to an embodiment of the present disclosure. The processor 502 may calculate the second circadian rhythm index Cir_idx_2 using a second algorithm according to an embodiment of the present disclosure.

The first algorithm may be defined as an algorithm for calculating the first circadian rhythm index using a first parameter, a second parameter, and a third parameter. The second algorithm may be defined as an algorithm for calculating the second circadian rhythm index using the first parameter, the second parameter, and the third parameter. The first parameter may be defined as a timing related to light irradiation, the second parameter may be defined as a weight at a specific timing, and the third parameter may be defined as a circadian illuminance value (or a standard circadian illuminance value). As another example, the second parameter may be defined as a light irradiation duration time.

In one embodiment, the first parameter may be a timing t at which light irradiation (or light exposure) from external light to the user's human body begins. The second parameter may be a weight at a specific timing t.

In another embodiment, the first parameter may be a timing t at which light irradiation (or light exposure) from external light to the user's human body begins. The second parameter may be a time d at which light is irradiated (or light is exposed) to the user's body from external light. As another example, the first parameter may be a timing (t′=t+d/2) corresponding to a median time of light irradiation (or light exposure) of the user's body from external light, that is, a median time of light irradiation (or light exposure).

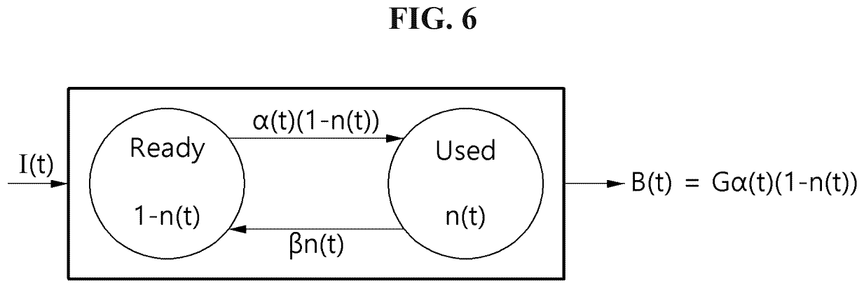

Detailed contents of the first algorithm for calculating the first circadian rhythm index Cir_idx_1 and the second algorithm for calculating the second circadian rhythm index Cir_idx_2 are described below in FIG. 6.

The processor 502 may calculate a light pollution index based on the first circadian rhythm index Cir_idx_1. The light pollution index may be defined as an indicator indicating a percentage of a degree at which a user's circadian rhythm is out of a normal circadian rhythm by external light to which the user is exposed for a certain time. The light pollution index may be determined using a value of the CPS, and be determined as a value between 0% and 100% so that the user may intuitively check the light pollution index at a glance. The more a user's circadian rhythm deviates from the normal circadian rhythm, resulting in a larger positive CPS value, the closer the light pollution index may be to 100%. The closer the user's circadian rhythm is to the normal circadian rhythm, resulting in a closer CPS value to 0, the closer the light pollution index may be to 0%.

The processor 502 may calculate a sleep disturbance index based on the second circadian rhythm index Cir_idx_2. The sleep disturbance index may be defined as an indicator indicating a user's predicted sleep quality due to the external light to which the user is exposed for a certain time. The sleep disturbance index is determined using the value of MSR, and can be determined as a value greater than 0 so that users may intuitively check the sleep disturbance index at a glance. That is, as the user's melatonin secretion is suppressed more than the normal melatonin secretion, the MSR has a larger value, so the sleep disturbance index may have a larger positive value. Conversely, as the user's melatonin secretion is closer to the normal melatonin secretion, the MSR has a smaller value, so the sleep disturbance index may have a value close to 0.

The memory 503 may store data required for the operation(s) performed by the circadian rhythm analysis device 500. As an example, the memory 503 may store the circadian illuminance value Cir_lx and/or the visual illuminance value Vis_lx. The memory 503 may store the first circadian rhythm index Cir_idx_1 and/or the second circadian rhythm index Cir_idx_2. The memory 503 may store the light pollution index and/or the sleep disturbance index. The memory 503 may store a timing related to light irradiation and/or a weight at a specific timing. The memory 503 may store data related to the circadian illuminance Cir_lx, a weight in a specific timing, and data related to a light irradiation time as a data set. The memory 503 may store a program configured to analyze a circadian rhythm.

As another example, the memory 503 may store a timing related to light irradiation and/or a light irradiation duration time. The memory 503 may store the data related to the circadian illuminance Cir_lx and the data related to a light irradiation time as a data set.

The memory 503 may include, but is not limited to, a volatile memory such as DRAM or SRAM, nonvolatile memory such as PRAM, MRAM, ReRAM, or NAND flash memory, or a hard disk drive (HDD) or a solid state drive (SSD). Further, the memory 503 may be a cache, a buffer, a main memory, an auxiliary memory, or a separately provided storage system depending on a purpose/location, but is not limited thereto.

FIG. 6 is a conceptual diagram of a model utilized in a first algorithm and a second algorithm according to an embodiment of the present disclosure.

Referring to FIG. 6, a result B(t) modeling an influence of a circadian illuminance value I(t) on a circadian rhythm may be confirmed. I(t) as an intensity of light at a time t may be defined as a circadian illuminance value. B(t) may be defined as a force which affects a circadian rhythm at a time t.

All light receptors present in a person may include a receptor which is in a state to react to light (i.e., a ready state) and a receptor which is in a state to already react to the light (i.e., a used state). A ratio of the receptor in the used state among the entirety and the receptor may be n(t), and a ratio of the receptor in the ready state among the entirety and the receptor may be represented by (1−n(t)).

α(t) may be defined as a ratio at which the state of the light receptor is changed from the ready state to the used state per hour. α(t) may also be expressed as α(I), as a function for I(t), i.e., the third parameter.

In an embodiment, the ratio at which the state of the light receptor is changed from the ready state to the used state may be a constant (β) rather than a function of t. As an example, β may be, but is not limited to, 0.42 per hour.

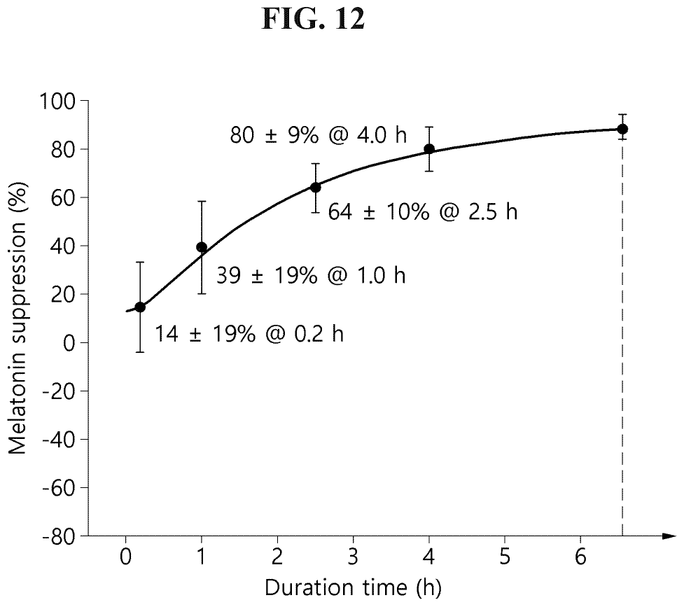

The receptor in the ready state can be represented by α(t)(1−n(t)), and the receptor in the used state may be represented as βn(t). Referring to FIG. 12, the differential equations for the models utilized in the first algorithm and the second algorithm may be expressed as Equation 1 below.

$$\frac{d n}{d t} = α ( t ) ( 1 - n ( t ) ) - β n ( t ) \qquad [ Equation 1 ]$$

By solving a differential equation of the model according to Equation 1, n(t) may be calculated. In an embodiment, it may be assumed that the circadian illuminance value remains constant for one hour. That is, it may be assumed that a(t)=α (α is a constant). Under this assumption, the solution of the differential equation can be expressed as Equation 2 below.

$$n ( t ) = ( n ( 0 ) - \frac{{α}_{f}}{{α}_{f} + β} ) {e}^{- ( {α}_{f} + β ) t} + \frac{{α}_{f}}{{α}_{f} + β} \qquad [ Equation 2 ]$$

In Equation 2, n(0), n(1), n(2), . . . , and n(24) may all be calculated assuming that the circadian illuminance value is given for each time period for 24 hours. Through a process of repeatedly obtaining the solutions of the differential equations for the models used in the first algorithm and the second algorithm, it is possible to find n(0) values satisfying n(0)=n (24).

The result (B(t)) of modeling the influence of the circadian illuminance value (I(t)) on the circadian rhythm may be expressed as Equation 3 below.

$$B ( t ) G α ( t ) ( 1 - n ( t ) ) \qquad [ Equation 3 ]$$

In Equation 3, G is a constant, and B(t) is in a linear relationship with the first or second circadian rhythm index.

An approximate expression of α(I) may be expressed as Equation 4 below.

$$α ( I ) = A + \frac{( K - A )}{1 + {( I / {I}_{0} )}^{p 2}} \qquad [ Equation 4 ]$$

In Equation 4, I is the circadian illuminance, and K, A, I0, and p2 are constants, respectively. In an embodiment, A may be about 6.53, K may be about 0.051, I0 may be about 403.267, and p2 may be about 1.439 when obtaining the first circadian rhythm index. In one embodiment, A may be about 5.613, K may be about −0.006, I0 may be about 121.469, and p2 may be about 3.577 when obtaining the second circadian rhythm index.

The circadian rhythmic index (i.e., the first circadian rhythm Index or the second circadian rhythm index) Cir calculated using B(t) may be expressed by Equation 5 below.

$$Cir = {\int }_{0}^{24} w ( t ) B ( t ) dt = {\int }_{0}^{24} w ( t ) G α ( t ) ( 1 - n ( t ) ) dt \qquad [ Equation 5 ]$$

In Equation 5, w(t) is a weight at a specific timing t, i.e., a second parameter.

Assuming that the circadian illuminance value remains constant for 1 hour, i.e., assuming that a(t)=α (α is a constant), the circadian rhythm index (i.e., the first circadian rhythm index or the second circadian rhythm Index) Cir calculated using B(t) may be expressed as in Equation 6 below.

$$Cir = {\sum }_{h = 1}^{24} {w}_{h} {B}_{s} ( h ) \qquad [ Equation 6 ]$$

In Equation 6, Wh is an average weight at each time interval of a predetermined time unit. Assuming that the predetermined time is 1 hour, Wh may be an average weight between (h−1) and h. BS(h) may be expressed as in Equation 7 below.

$${B}_{s} ( h ) = \frac{1}{G} {\int }_{h - 1}^{h} B ( t ) dt = {\int }_{h - 1}^{h} α ( t ) ( 1 - n ( t ) ) dt \qquad [ Equation 7 ]$$

Wh may have a different value for each of the first and second circadian rhythm indices. In addition, the Wh of the first or second circadian rhythm indices may be different values from time to time. As an example, the W1 of the first circadian rhythm index may be about 1.142, and the W2 of the first circadian rhythm index may be about 1.11. As an example, the W1 of the second circadian rhythm index may be about 0.373, and the W2 of the second circadian rhythm index may be about 0.362.

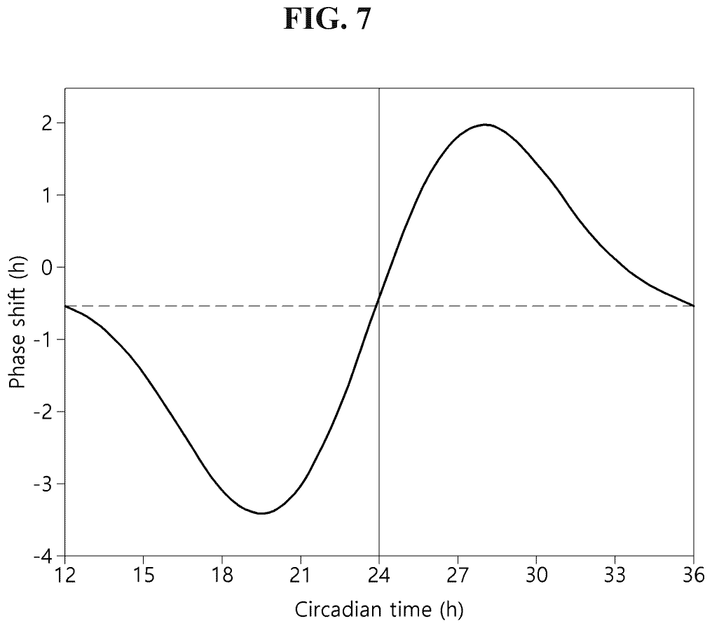

FIG. 7 and below illustrate graphs showing results of clinical experiments performed under a predetermined condition. Specifically, referring to the graphs of FIGS. 7 to 10, a circadian phase shift (CPS) value corresponding to an index indicating the degree of shift in the phase of the circadian rhythm, i.e., a first circadian rhythm index, may be derived.

The result values may vary if the predetermined conditions are different, and the predetermined conditions do not limit a technical scope of the present disclosure. Clinical experiments are experiments on human light exposure using lighting with specific photometric characteristics (e.g., a color temperature and a color rendering index (CRI)), and are conducted to measure changes in the circadian rhythm which affect a human body with only items related to external light (e.g., light exposure time and interval) as variables. As an example, a lighting with a color temperature of 4100 K and a color rendering index of 85 may be used.

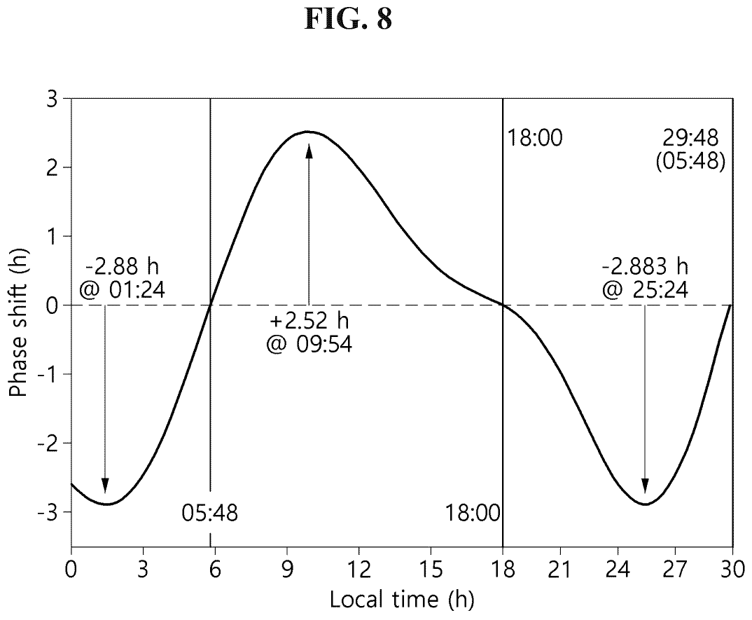

FIG. 7 is a graph showing a phase shift value for a circadian time according to an embodiment of the present disclosure. FIG. 8 is a graph showing a phase shift value for a local time according to an embodiment of the present disclosure.

FIGS. 7 and 8 illustrate graphs of clinical experimental data for deriving a phase shift value for a first parameter. In other words, FIGS. 7 and 8 illustrate graphs showing results of deriving a correlation between a timing related to light irradiation and a phase shift value of the circadian rhythm through a clinical experiment. The timing related to the light irradiation may be a circadian time or a local time.

Referring to FIG. 7, a phase shift value (h) according to a circadian time (h) is illustrated. The circadian time may be defined as a relative time based on a core body temperature (CBT) minimum time. The circadian time may be referred to as circadian phase, and a time when a core body temperature is the lowest may be defined as circadian time 0 h. In an embodiment, a phase shift value from circadian time 12 h to 36 h may have a value according to a sine function waveform in time series, as illustrated in FIG. 7.

Referring to FIG. 8, a phase shift (h) value according to the local time (h) is illustrated. The local time may be defined as a local time of an area being discussed when talking about a time of occurrence of an event. In some embodiments, the local time may be a clock time. A local time, which is an x-axis variable in FIG. 8, is an absolute time, unlike the circadian time in FIG. 7.

A melatonin secretion start point (dim light melatonin onset; DLMOn) may be calculated based on a graph fitting results of clinical experiments to determine when melatonin begins to be secreted, when the melatonin begins to be suppressed, and when the melatonin has a peak value. According to the clinical experiment results, an x axis of a phase response curve may be converted from the circadian time (see FIG. 7) to the local time (see FIG. 8). The melatonin secretion onset point (DLMOn) may be determined by measuring melatonin concentration and observing a point of increase. According to an embodiment, the melatonin secretion onset point (DLMOn) may be calculated as a value corresponding to 25% of a peak value of melatonin secretion. Based on an absolute timing corresponding to a predetermined melatonin secretion onset point (DLMOn) as a result of the clinical experiment, a circadian phase shift (CPS) value may be calculated by the first to third parameters.

Referring to FIG. 8, after 05 h 48 m, a phase advance may occur in the circadian rhythm, and after 18 h, a phase delay may occur in the circadian rhythm.

A formula may be derived to calculate the phase shift value using the timing related to the light irradiation as a variable. As an example, a phase shift value y₁ with a timing t when the user's body is exposed to light from external light as a variable may be expressed as in Equation 8 below.

$${y}_{1} = {A}_{1} cos ( 0.0833 π t + 1.0153 π ) + {A}_{2} cos ( 0.1667 π t + 0.582 π ) {A}_{3} \qquad [ Equation 8 ]$$

Referring to Equation 8, A₁ and A₂ are each factors, and A₃ is a constant. A₁, A₂, and A₃ may vary depending on predetermined conditions of the clinical experiment, such as a user's surrounding environment, etc. For example, A₁ may be 2.2794, A₂ may be 0.8306, and A₃ may be −0.1019.

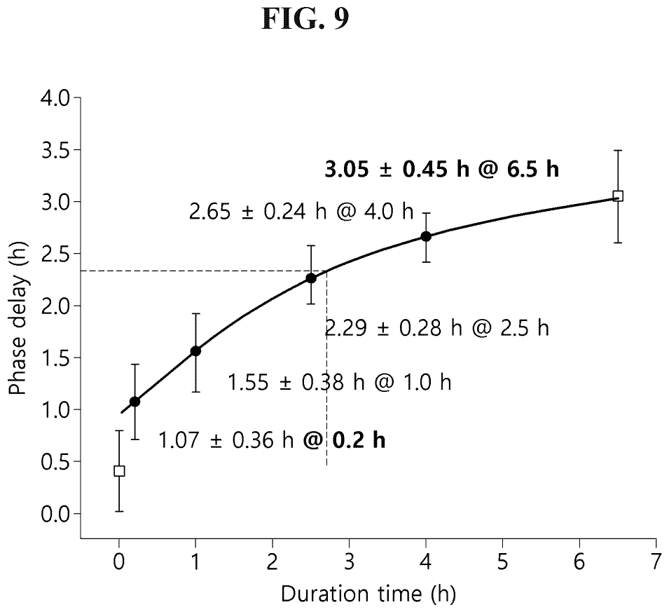

FIG. 9 is a graph showing a phase shift value with respect to light irradiation duration according to an embodiment of the present disclosure.

FIG. 9 illustrates a graph of clinical experimental data for deriving a phase shift value for a second parameter. In other words, FIG. 9 illustrates a graph showing a result of deriving a correlation between the light irradiation duration time and the phase shift value the circadian rhythm through a clinical experiment.

Referring to FIG. 9, a phase shift (h) value according to a light irradiation duration time (h) is illustrated. As the light irradiation duration time increases, the phase shift value of the circadian rhythm may increase. As an example, when light irradiation continues for 0.2 h, a phase delay of (1.07±0.36) h may occur in the circadian rhythm.

A formula may be derived to calculate the phase shift value using the light irradiation duration time as a variable. A phase shift value y₂ with a light irradiation duration time d as a variable may be expressed as in Equation 9 below.

$${y}_{2} = {A}_{4} + \frac{{A}_{5} - {A}_{6}}{1 + {( d / {d}_{1} )}^{p 1}} \qquad [ Equation 9 ]$$

Referring to Equation 9, A₄, A₅, A₆, d₁, and p₁ are each constants. A₄, A₅, A₆, d₁, and p₁ may vary depending on predetermined conditions of the clinical experiment, such as a user's surrounding environment, etc. d may be defined as a duration time for which the user is exposed to light. For example, A₄ may be 3.69, As may be 0.97, A₆may be 3.69, d₁ may be 2.69, and p₁ may be 1.28.

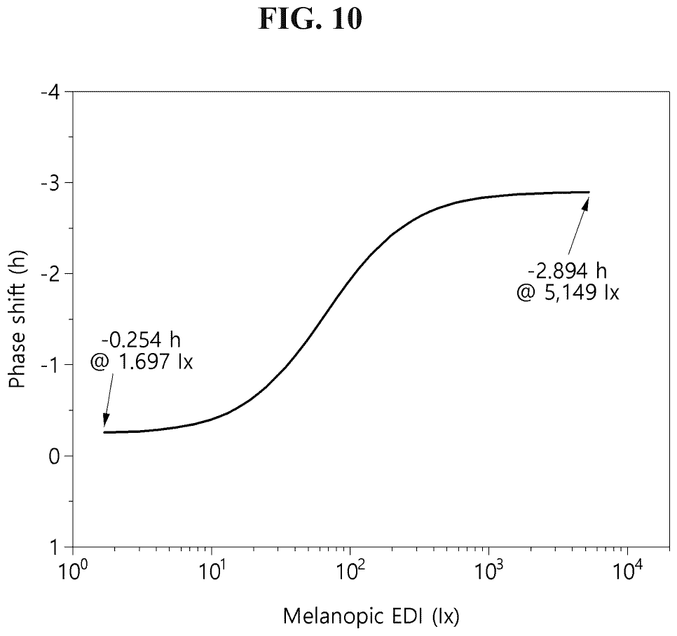

FIG. 10 is a graph showing a phase shift value for a circadian luminance according to an embodiment of the present disclosure.

FIG. 10 illustrates a graph of clinical experimental data for deriving a phase shift value for a third parameter. In other words, FIG. 10 illustrates a graph showing a result of deriving a correlation between a standard circadian illuminance and the phase shift value of the circadian rhythm through a clinical experiment.

In the clinical experiment, a phase shift value (h) according to a visual illuminance (VIL) may be derived. In an embodiment, a correlation between VIL and a phase shift value for a light irradiation duration time of 6.5 h may be derived. According to an embodiment of the present disclosure, the VIL may be converted into melanopic equivalent daylight illuminance (EDI) (lx) using a predetermined melanopic daylight efficiency ratio (DER). The VIL may be defined as the visual illuminance. The melanopic DER as an abbreviation of a melanopic daylight efficacy ratio may be defined as a spectral metric of a biological effectiveness of a particular artificial light source compared to 6500 K daylight. The melanopic DER is an indicator indicating how efficiently a particular artificial light source may regulate melatonin under the same illuminance as a standard light source at 6500 K. The melanopic DER may be determined by a spectral distribution of a light source. In an embodiment, the standard circadian illuminance may be a melanopic EDI. In an embodiment, the melanopic DER may be determined as 0.566. The melanopic EDI may be calculated as a product of the VIL and the melanopic DER.

Referring to FIG. 10, a phase shift value (h) according to a melanopic equivalent daylight illuminance (EDI) (lx) is illustrated. As an example, when the melanopic EDI is 1.697 lx, a phase delay of 0.254 h may occur in the circadian rhythm.

In other words, when the melanopic EDI is 1.697 lx, the phase shift value in the circadian rhythm may be −0.254 h.

A formula may be derived to calculate the phase shift value using the standard circadian illuminance value, i.e., the melatopic EDI value as a variable. A phase shift value y₃ with a melatonin EDI value Eₘₑₗ as a variable may be expressed as in Equation 10 below.

$${y}_{3} = {A}_{7} + \frac{{A}_{6} - {A}_{9}}{1 + {( {E}_{mel} / {E}_{1} )}^{p 2}} \qquad [ Equation 10 ]$$

Referring to Equation 10, A₇, A₈, A₉, E₁, and p₂ are each a constant. A₇, A₈, A₉, E₁, and p₂ may vary depending on predetermined conditions of the clinical experiment, such as a user's surrounding environment, etc. For example, A₇ may be −2.9, A₈ may be −0.24, A₉ may be −2.9, E₁ may be 67.33, and p₂ may be 1.42.

In FIGS. 7 to 10, correlations between the first to third parameters and the circadian phase shift (CPS) may be calculated, respectively. A circadian phase shift value according to the first parameter, a circadian phase shift value according to the second parameter, and a circadian phase shift value according to the third parameter may be converted and standardized so that data matches at the same reference point. For example, the same reference point may be a range of timing related to light irradiation, a range of a light irradiation duration time, and/or a range of a melanopic EDI.

For example, Equation 11 be expressed as follows by converting and standardizing Equation 8 as the same reference point.

$${y}_{1} = 2.2794 cos ( 0.0833 π {t}^{′} + 1.0153 π ) + 0.8306 cos ( 0.1667 π {t}^{′} + 0.582 π ) + 0.1019 \qquad [ Equation 11 ]$$

Referring to Equation 11, t′ is a timing corresponding to a median value of the time d during which light is irradiated (or exposed to light) from external light to the user's body. Equation 11 adopts a conversion equation (t′=t+d/2) for converting a timing t at which light irradiation (or light exposure) to the user's body from external light starts into a light irradiation median time t′.

Equation 12 may be expressed as follows by converting and standardizing Equation 9 as the same reference point.

$${y}_{2} = 1.2055 + \frac{0.3391 - 1.2055}{1 + {( d / 2.69 )}^{1.28}} \qquad [ Equation 12 ]$$

Equation 13 may be expressed as follows by converting and standardizing Equation 10 to the same reference point.

$$CPS -- ( a ) \times ( b ) \times ( c ) \qquad [ Equation 13 ]$$

According to the first algorithm of an embodiment of the present disclosure, the first circadian rhythm index (or, circadian phase shift (CPS)) (h) may be expressed as in Equation 14 below.

$$CPS -- ( a ) \times ( b ) \times ( c ) \qquad [ Equation 14 ]$$

Referring to Equation 14, Equations 11 to 13, which have the same range of timing related to light irradiation, the same range of light irradiation duration time, and the same range of melanopic EDI, may be (a), (b), and (c), respectively. Equations (a), (b), and (c) may be integrated into a single equation as in Equation 14. When the CPS has a negative value, it means that the phase delay occurs in the circadian rhythm, and when CPS has a positive value, it means that the phase advance occurs in the circadian rhythm.

In an embodiment, when the timing (t′=t+d/2) corresponding to the median value of the time d during which light is irradiated to the user's body from external light is 20 h, a duration time d of the light irradiation is 6 h, and the circadian illuminance value (or melatopic EDI value) is 500 lx, the CPS value may be calculated as 0.496 h using Equation 14. The CPS value may be calculated by setting the light irradiation duration time d to be 6 h, that is, from 17 h to 23 h.

In an embodiment, when the timing (t′=t+d/2) corresponding to the median value of the time d during which light is irradiated to the user's body from external light is 21.5 h, a duration time d of the light irradiation is 4.5 h, and the circadian illuminance value (or melatopic EDI value) is 273 lx, the CPS value may be calculated as 1.017 h using Equation 14.

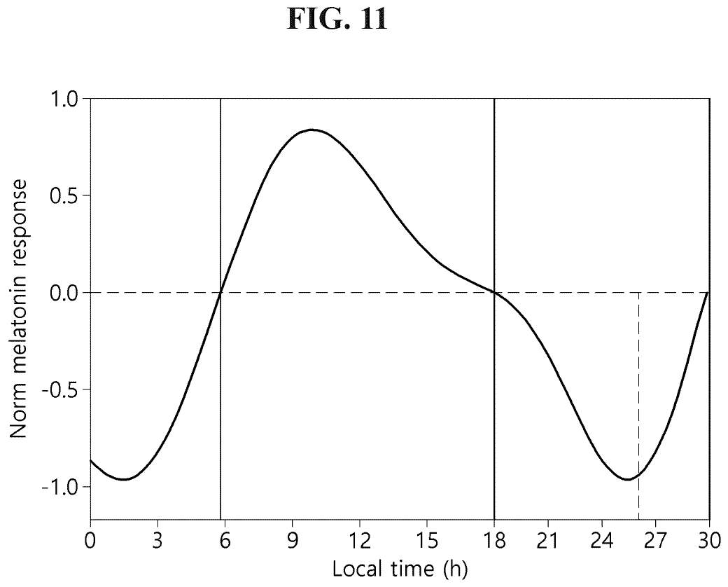

FIGS. 11 to 13 illustrate graphs showing results of clinical experiments performed under a predetermined condition. Specifically, referring to the graphs of FIGS. 11 to 13, a melatonin suppression response (MSR) value corresponding to an indicator indicating the degree of melatonin secretion suppression, i.e., a second circadian rhythm index, may be derived. The MSR, like CPS value, may be calculated by the first to third parameters.

FIG. 11 illustrates a graph showing a normalized melatonin response with respect to a local time according to an embodiment of the present disclosure. In other words, FIG. 11 illustrates a graph showing a result of deriving a correlation between a timing related to light irradiation and a melatonin response through a clinical experiment. The timing related to the light irradiation may be a circadian time or a local time.

Hereinafter, description is given with reference to FIGS. 7 and 8, and duplicated contents are omitted.

Referring to FIGS. 8 and 11, in a phase response curve (see FIG. 8) where an x axis is the local time (h), a y value may be converted to a melatonin response (norm melatonin response) curve normalized from −1 to 1 (or from −100% to 100%).

According to the clinical experiment results, the x axis of the melatonin response curve may be converted from the circadian time (see FIG. 7) to the local time (see FIG. 11). Content identical or similar to FIG. 8 are omitted with respect to conversion into the local time. Based on an absolute time corresponding to a predetermined melatonin secretion onset point (DLMOn) as a result of the clinical experiment, a melatonin suppression response (MSR) value may be calculated by the first to third parameters.

A formula may be derived to calculate the melatonin suppression value using the timing related to the light irradiation as a variable. As an example, a melatonin suppression value y₄ with a timing t when the user's body is exposed to light from external light as a variable may be expressed as in Equation 15 below.

$${y}_{4} = {A}_{10} cos ( 0.0833 π t + 1.0153 π ) + {A}_{11} cos ( 0.1667 π t + 0.582 π ) {A}_{12} \qquad [ Equation 15 ]$$

Referring to Equation 15, A₁₀ and A₁₁ are each a factor, and A₁₂ is a constant. A₁₀, A₁₁, and A₁₂ may vary depending on predetermined conditions of the clinical experiment, such as a user's surrounding environment, etc.

FIG. 12 is a graph showing a melatonin suppression value with respect to the light irradiation duration according to an embodiment of the present disclosure.

FIG. 12 illustrates a graph of clinical experimental data for deriving a melatonin suppression value for a second parameter. In other words, FIG. 9 illustrates a graph showing a result of deriving a correlation between the light irradiation duration time and a melatonin value through a clinical experiment.

Referring to FIG. 12, a melatonin suppression value (%) according to the light irradiation duration time (h) is illustrated. As the light irradiation duration time increases, the melatonin suppression value may increase. As an example, when light irradiation continues for 0.2 h, melatonin may be suppressed by (14±19)%.

A formula may be derived to calculate the melatonin suppression value using the light irradiation duration time as a variable. A melatonin suppression value y₅ with a light irradiation duration time d as a variable may be expressed as in Equation 16 below.

$${y}_{5} = {A}_{13} + \frac{{A}_{14} - {A}_{15}}{1 + {( d / {d}_{2} )}^{p 3}} \qquad [ Equation 16 ]$$

Referring to Equation 16, A₁₃, A₁₄, A₁₅, d₂, and p₃ are each constants. A₁₃, A₁₄, A₁₅, d₂, and p₃ may vary depending on predetermined conditions of the clinical experiment, such as a user's surrounding environment, etc. d may be defined as a duration time for which the user is exposed to light. For example, A₁₃ may be 100, A₁₄ may be 12.44, A₁₅ may be 100, d₂ may be 1.92, and p₃ may be 1.55.

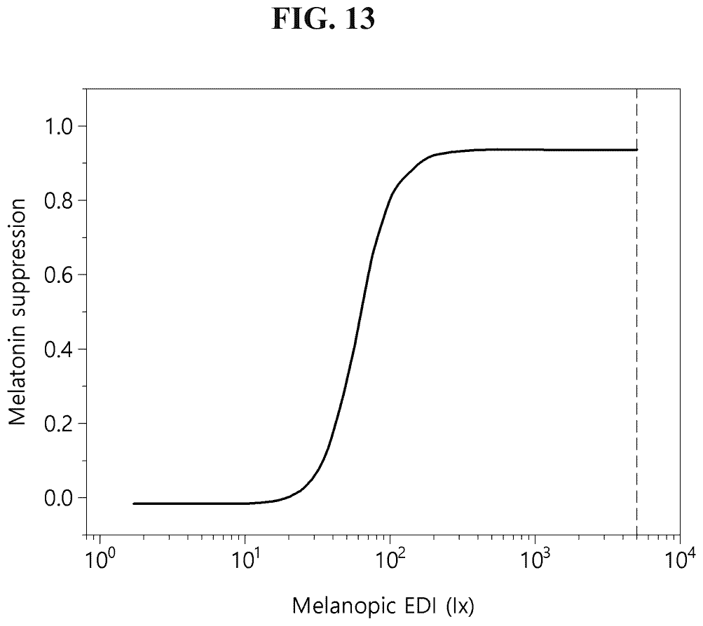

FIG. 13 is a graph showing the melatonin suppression value with respect to the circadian luminance according to an embodiment of the present disclosure.

FIG. 13 illustrates a graph of clinical experimental data for deriving a melatonin suppression value for a third parameter. In other words, FIG. 13 illustrates a graph showing a result of deriving a correlation between a standard circadian illuminance and the melatonin suppression value through a clinical experiment.

In the clinical experiment, a melatonin suppression value according to a visual illuminance (VIL) may be derived. In an embodiment, a correlation between the VIL and the melatonin suppression value for a light irradiation duration time of 6.5 h may be derived. Contents identical or similar to FIG. 10 with respect to converting VIL to melanopic EDI are omitted. Referring to FIG. 13, the melatonin suppression value according to the melanopic equivalent daylight illuminance (EDI) (lx) is illustrated.

A formula may be derived to calculate the melatonin suppression value using the circadian standard illuminance value, i.e., the melatopic EDI value, as a variable. The melatonin suppression value y₆ with the melatopic EDI value Eₘₑₗ as a variable may be expressed as in Equation 17 below.

$${y}_{6} = {A}_{16} + \frac{{A}_{17} - {A}_{18}}{1 + {( {E}_{mel} / {E}_{2} )}^{p 4}} \qquad [ Equation 17 ]$$

Referring to Equation 17, A₁₆, A₁₇, A₁₈, d₂, and p₄ are each a constant. A₁₆, A₁₇, A₁₈, E₂, and p₄ may vary depending on predetermined conditions of the clinical experiment, such as a user's surrounding environment, etc. For example, A₁₆ may be 0.936, A₁₇ may be −0.0156, A₁₈ may be 0.936, E₂ may be 59.975, and p₄ may be 3.55.

In FIGS. 11 to 13, correlations between the first parameter to the third parameter and melatonin suppression response (MSR) value may be calculated, respectively. A circadian melatonin suppression value according to the first parameter, a melatonin suppression value according to the second parameter, and a melatonin suppression value according to the third parameter may be converted and standardized so that data matches each other at the same reference point. For example, the same reference point may be a range of timing related to light irradiation, a range of a light irradiation duration time, and/or a range of an melanopic EDI.

According to an embodiment of the present disclosure, the melatonin suppression value may be converted and normalized so that the melatonin suppression values according to the first parameter and the third parameter match, each other based on the second parameter being 6.5 h.

For example, Equation 18 be expressed as follows by converting and standardizing Equation 15 to the same reference point.

$${y}_{4} = 0.8267 cos ( 0.0 \; 833 π {t}^{′} + 1.0153 π ) + 0.3 012 cos ( 0.1667 π {t}^{′} + 0.534 π ) + 0.0379 \qquad [ Equation 18 ]$$

Referring to Equation 18, t′ is a timing corresponding to a median value of the time d during which light is irradiated (or exposed to the user's body) from external light to the user's body. Equation 18 adopts a conversion equation (t′=t+d/2) for converting a timing t at which light irradiation (or light exposure) to the user's body from external light starts into a light irradiation median time t′.

Equation 19 may be expressed as follows by converting and standardizing Equation 16 to the same reference point.

$${y}_{5} = 1 + \frac{0.1 \; 244 - 1}{1 + {( d / 1.92 )}^{1.55}} \qquad [ Equation 19 ]$$

Equation 20 may be expressed as follows by converting and standardizing Equation 17 to the same reference point.

$${y}_{6} = 1 + \frac{- 1}{1 + {( {E}_{mel} / 59.975 )}^{3.55}} \qquad [ Equation 20 ]$$

According to the second algorithm of an embodiment of the present disclosure, the second circadian rhythm index (or, melatonin suppression response (MSR)) (%) may be expressed as in Equation 21 below.

$$MSR - ( d ) \times ( e ) \times ( f ) \qquad [ Equation 21 ]$$

Referring to Equation 21, Equations 18 to 20, which have the same range of timing related to light irradiation, the same range of light irradiation duration time, and the same range of melanopic EDI, may be (d), (e), and (f), respectively. Equations (d), (e), and (f) may be integrated into a single equation as in Equation 21. When the MSR has a negative value, it means melatonin suppression (or melatonin secretion is inhibited), and when the MSR has a positive value, it means melatonin secretion (or melatonin is secreted). Melatonin suppression means that melatonin secretion is suppressed, so that melatonin secretion is relatively low, and melatonin secretion means that melatonin secretion is relatively less suppressed, so that melatonin secretion is relatively much.

In an embodiment, when the timing (t′=t+d/2) corresponding to the median value of the time d during which light is irradiated to the user's body from external light is 20 h, a duration time d of the light irradiation is 6 h, and the circadian illuminance value (or melatopic EDI value) is 500 lx, the MSR value may be calculated as −17.06% using Equation 21. The MSR value may be calculated by setting the light irradiation duration time d to be 6 h, that is, from 17 h to 23 h. As another example, by setting the light irradiation duration time d to be 1 h from 17 h to 18 h, 18 h to 19 h, 19 h to 20 h, 20 h to 21 h, 21 h to 22 h, and 22 h to 23 h, that is, for 1 h, and adding up the six calculated CPS values, the MSR value may be calculated when the light irradiation duration time d is 6 h. In addition to two methods described above, various CPS calculation methods may be applied under the same reference point, and these methods do not limit the technical scope of the present disclosure.

In another embodiment, when the time (t′=t+d/2) corresponding to the median value of the time d during which light is irradiated to the user's body from external light is 21.5 h, a duration time d of the light irradiation is 4.5 h, and the circadian illuminance value (or melanopic EDI value) is 273 lx, the MSR value may be calculated as −37.12% using Equation 21.

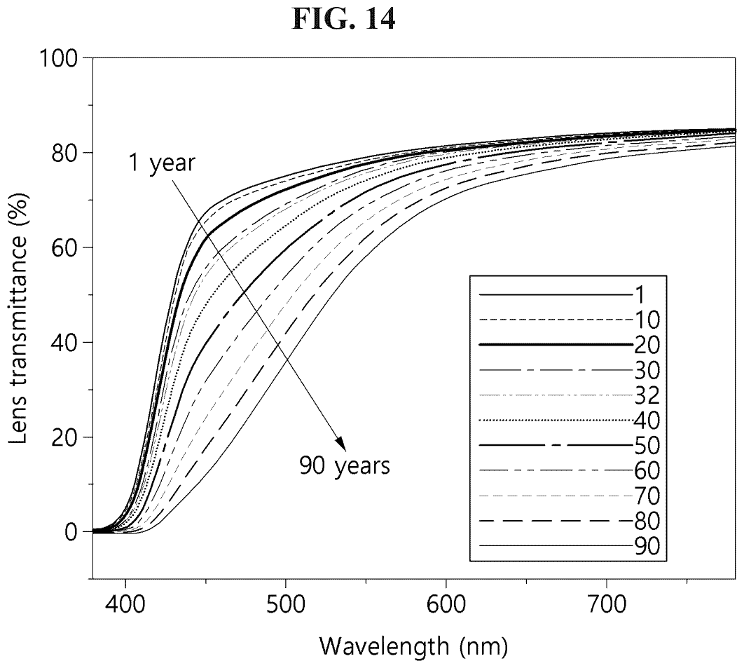

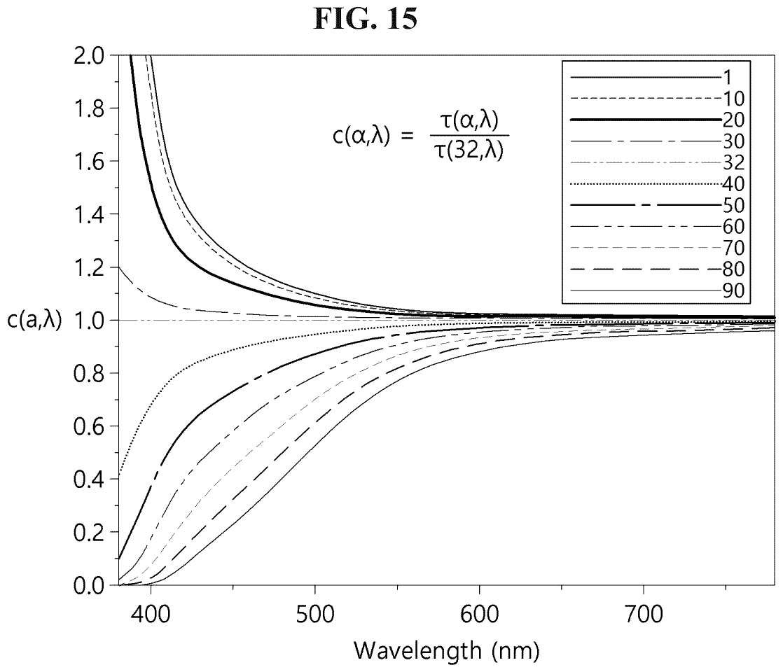

FIG. 14 is a graph showing lens transmittance according to a wavelength by each user's age according to an embodiment of the present disclosure. FIG. 15 is a graph showing a ratio of lens transmittance by wavelength compared to a 32-year-old user according to an embodiment of the present disclosure.

A standard for the melanopic equivalent daylight illuminance (EDI) (lx) is established based on a 32-year-old user. For users other than 32-year-old users, lens transmittance correction according to age is required. In an embodiment, the circadian illuminance (or standard circadian illuminance may be the melanopic EDI.

Referring to FIG. 14, a wavelength-specific lens transmittance for each user from 1 year to 90 years of age is illustrated. As the user ages, lens transmittance may decrease. A lens transmittance close to 100% may mean that the user is receiving all of the light of the corresponding wavelength. In particular, lens transmittance shows a large difference depending on age, especially in blue light transmittance. As an example, even when exposed to the same amount of light as melanopic EDI 100 lx, older people receive a smaller amount of light.

A lens transmittance, τ(α, λ), may be expressed as in Equations 22 and 23 below.

$$τ ( α , λ ) = 1 {0}^{- {D}_{τ} ( α , / λ )} \qquad [ Equation 22 ]$$

$${D}_{τ} ( α , λ ) = 0.06 + ( 0.5 + 3.1 \times 1 {0}^{- 5} {a}^{2} ) {( \frac{400}{λ} )}^{4} + 151.5492 {e}^{{( - 0.057 ( λ - 273 ) )}^{2}} + 2.13 ( 1 \text{.05} - 6.3 \times {10}^{- 5} {a}^{2} ) {e}^{{( - 0.029 ( λ - 370 ) )}^{2}} + 11.95 ( 0.059 + 1 \text{.86} \times ( 1 {0}^{- 4} {a}^{2} ) {e}^{{( - 0.021 ( λ - 325 ) )}^{2}} + 1.43 ( 0.016 + 1 \text{.32} \times 1 {0}^{- 4} {a}^{2} ) {e}^{( - {( 0.008 ( λ - 325 ) )}^{2}} \qquad [ Equation 23 ]$$

Referring to Equations 22 and 23, a represents the user's age and λ represents a wavelength of light.

The ratio of lens transmittance compared to 32 years old, C(α, λ), may be expressed as in Equation 24 below.

$$c ( α , λ ) = \frac{τ ( α , λ )}{τ ( 32 , λ )} \qquad [ Equation 24 ]$$

Age corrected melanopic EDI according to the lens transmittance, Ε_{v,mel}^{D65} (α) may be expressed as in Equation 25 below.

$${E}_{v , mel}^{D 6 {5}_{e}} ( α ) = {E}_{v , mel}^{D 65} \times {k}_{mel , τ} ( α ) \qquad [ Equation 25 ]$$

Referring to Equation 25, K_{mel,λ}(α) means an age-related transmittance ratio of melanopic efficacy.

K_{mel,λ}(α) may be expressed as in Equation 26 below.

$${h}_{mel , τ} ( α ) = \frac{{\int }_{380}^{780} {E}_{e , λ} ( λ ) c ( α , λ ) {S}_{mel} ( λ ) d λ}{{\int }_{350}^{780} {E}_{e , λ} ( λ ) {S}_{mel} ( λ ) d λ} \qquad [ Equation 26 ]$$

In Equation 26, Sₘₑₗ(λ) means the melatonin sensitivity curve and Ε_{e,λ} (λ) represents the spectrum curve of the light source.

In an embodiment, each age-specific preset of each light source in which a spectrum power distribution (SPD) is known may be configured, such that K_{mel,τ}(α) may be provided. When a value of K_{mel,τ}(α) according to the user's age and light source is determined, age-reflecting melanopic EDI may be calculated.

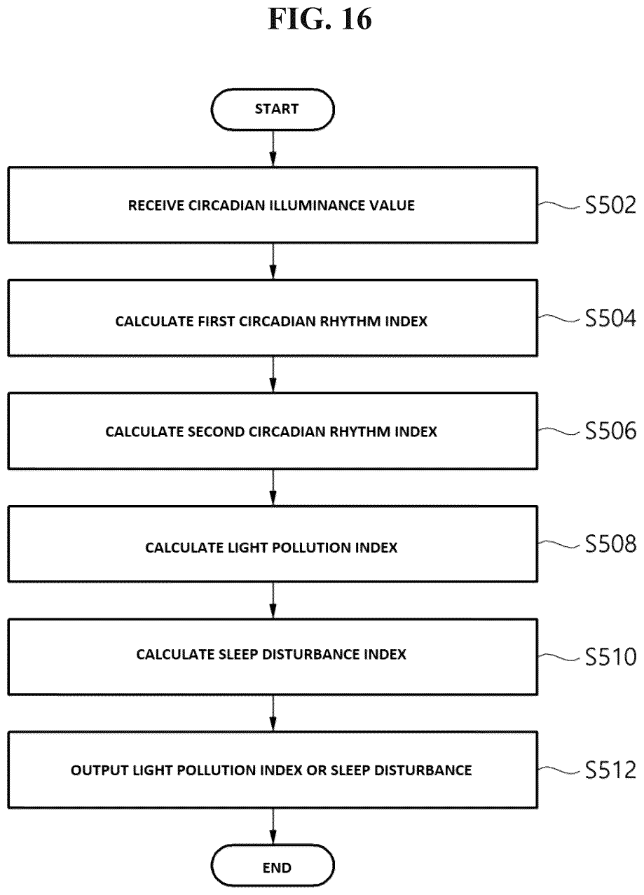

FIG. 16 is a flowchart showing a method for analyzing a circadian rhythm index of a circadian rhythm analysis device according to an embodiment of the present disclosure.

In S502, the circadian rhythm analysis device may receive a circadian illuminance values from the outside. The circadian rhythm analysis device can receive A circadian illuminance value and/or A visual illuminance value from the circadian illuminance measurement device 400 in FIG. 4.

In S504, the circadian rhythm analysis device may calculate a first circadian rhythm index corresponding to an indicator indicating a degree of shift in a phase of the circadian rhythm, based on the circadian illuminance value. In an example, the first circadian rhythm index may be calculated by an integral equation (Equation 5 or 6) using a weight at a specific timing and the circadian illuminance value at a specific timing. In another example, the first circadian rhythm index may be a circadian phase shift (CPS) value calculated based on an absolute time corresponding to a predetermined dim light melatonin onset (DLMO).

In S506, the circadian rhythm analysis device may calculate a second circadian rhythm index corresponding to an indicator indicating a degree of melatonin secretion suppression based on the circadian illuminance value. In an example, the second circadian rhythm index may be calculated by an integral equation (Equation 5 or 6) using a weight at a specific timing and the circadian illuminance value at a specific timing. In another example, the second circadian rhythm index may be a melatonin suppression response (MSR) value calculated based on an absolute timing corresponding to a predetermined dim light melatonin onset (DLMO).

In an example, the first circadian rhythm index or the second circadian rhythm index is calculated by the first parameter, the second parameter and the third parameter, and the first parameter may be the timing related to light irradiation, the second parameter may be a weight at a specific timing, and the third parameter may be the circadian illuminance value.

In another example, the first circadian rhythm index or the second circadian rhythm index is calculated by the first parameter, the second parameter and the third parameter, and the first parameter may be the timing related to light irradiation, the second parameter may be the light irradiation duration time, and the third parameter may be the circadian illuminance value.

In S508, the circadian rhythm analysis device may calculate a light pollution index based on the first circadian rhythm index. The light pollution index may be an indicator indicating the degree of disruption of the circadian rhythm determined by external light.

In S510, the circadian rhythm analysis device may calculate a sleep disturbance index based on the second circadian rhythm index. The sleep disturbance index may be an indicator indicating a quality of sleep determined by the external light.

In S512, the circadian rhythm analysis device may output the light pollution index or the sleep disturbance index.

Hereinabove, embodiments are disclosed in the drawings and specifications. Although specific terms have been used to describe embodiments in this specification, they have been used only for the purpose of explaining the technical idea of the present disclosure and are not intended to limit the meaning or the scope of the present disclosure set forth in the claims. Therefore, it will be appreciated by those skilled in the art that various modifications and other embodiments equivalent thereto can be made therefrom. Therefore, the true technical scope of the present disclosure should be defined by the technical idea of the appended claims.

1. A device for analyzing a circadian rhythm index, the device comprising:
a transceiver configured to receive a circadian illuminance value; and
at least one processor configured to calculate a first circadian rhythm index corresponding to an indicator indicating a shift degree of a phase of a circadian rhythm based on the circadian illuminance value, and calculate a second circadian rhythm index corresponding to an indicator indicating a melatonin secretion suppression degree based on the circadian illuminance value,
wherein the first circadian rhythm index and the second circadian rhythm index are indicators indicating an influence of the circadian illuminance on the circadian rhythm, and
the first circadian rhythm index or the second circadian rhythm index is calculated by an integral equation using a weight at a specific timing and the circadian illuminance value at a specific timing.

2. The device for analyzing a circadian rhythm index of claim 1, wherein the processor is additionally configured to calculate a light pollution index based on the first circadian rhythm index, and the light pollution index is an indicator indicating a degree of disruption of the circadian rhythm determined by external light.

3. The device for analyzing a circadian rhythm index of claim 2, wherein the processor is additionally configured to calculate a sleep disturbance index based on the second circadian rhythm index, and
the sleep disturbance index is an indicator indicating a quality of sleep determined by the external light.

4. The device for analyzing a circadian rhythm index of claim 3, wherein the first circadian rhythm index or the second circadian rhythm index is calculated by a first parameter, a second parameter, and a third parameter, and
the first parameter is a timing related to light irradiation, the second parameter is a weight at the specific timing, and the third parameter is the circadian illuminance value.

5. The device for analyzing a circadian rhythm index of claim 4, wherein the first circadian rhythm index or the second circadian rhythm index is calculated by Equation 1,
$$Cir = {\int }_{0}^{24} w ( t ) B ( t ) dt = {\int }_{0}^{24} w ( t ) G α ( t ) ( 1 - n ( t ) ) dt \qquad [ Equation l ]$$
where w(t) represents the second parameter, G represents a constant, α(t) represents a ratio at which a state of a light receptor is changed from ready to used, and (1−n(t)) represents a ratio of light receptors in the ready state among all light receptors.

6. The device for analyzing a circadian rhythm index of claim 5, wherein n(t) is calculated by Equation 2,
$$n ( t ) = ( n ( 0 ) - \frac{{α}_{f}}{{α}_{f} + β} ) {e}^{- ( {α}_{i} + β ) t} + \frac{{α}_{f}}{{α}_{f} + β} \qquad [ Equation 2 ]$$
where β represents a ratio at which the state of the light receptor is changed from used to ready, and α represents a constant value of α(t) when the circadian illuminance value is maintained constantly during a predetermined period.

7. The device for analyzing a circadian rhythm index of claim 6, wherein when the predetermined period is 1 h,
α(t) or n(t) is calculated by using the third parameter value for each time which is a constant value, and
the first circadian rhythm index or the second circadian rhythm index is calculated by Equations 3 and 4,
$$Cir = {\sum }_{h = 1}^{24} {w}_{h} {B}_{5} ( h ) \qquad [ Equation 3 ]$$
$${B}_{s} ( h ) = \frac{1}{G} {\int }_{h - 1}^{b} B ( t ) d t = {\int }_{h - 1}^{h} α ( t ) ( 1 - n ( t ) ) dt \qquad [ Equation 4 ]$$
where h represents a timing of a 1 h unit, and Wh represents an average value of the second parameter between (h−1) and h.

8. The device for analyzing a circadian rhythm index of claim 3, wherein the first circadian rhythm index or the second circadian rhythm index is calculated by a first parameter, a second parameter, and a third parameter, and
the first parameter is the timing related to light irradiation, the second parameter is a light irradiation duration time, and the third parameter is the circadian illuminance value.

9. The device for analyzing a circadian rhythm index of claim 8, wherein the first circadian rhythm index is a circadian phase shift (CPS) value calculated based on an absolute timing corresponding to a predetermined dim light melatonin onset (DLMO).

10. The device for analyzing a circadian rhythm index of claim 9, wherein the second circadian rhythm index is a melatonin suppression response (MSR) value calculated based on an absolute timing corresponding to a predetermined dim light melatonin onset (DLMO).

11. The device for analyzing a circadian rhythm index of claim 10, wherein the transceiver is additionally configured to output the light pollution index or the sleep disturbance index.

12. An operation method for analyzing a circadian rhythm index performed by a circadian rhythm index analysis device, the method comprising:
receiving a circadian illuminance value;
calculating a first circadian rhythm index corresponding to an indicator indicating a degree of shift in a phase of a circadian rhythm based on the circadian illuminance value; and
calculating a second circadian rhythm index corresponding to an indicator indicating a melatonin secretion suppression degree based on the circadian illuminance value,
wherein the first circadian rhythm index and the second circadian rhythm index are indicators indicating an influence of the circadian illuminance on the circadian rhythm, and
the first circadian rhythm index or the second circadian rhythm index is calculated by an integral equation using a weight at a specific timing and the circadian illuminance value at a specific timing.

13. The operation method for analyzing a circadian rhythm index of claim 12, further comprising:
calculating a light pollution index based on the first circadian rhythm index,
wherein the light pollution index is an indicator indicating a degree of disruption of the circadian rhythm determined by external light.

14. The operation method for analyzing a circadian rhythm index of claim 13, further comprising:
calculating a sleep disturbance index based on the second circadian rhythm index,
wherein the sleep disturbance index is an indicator indicating a quality of sleep determined by the external light.

15. The operation method for analyzing a circadian rhythm index of claim 14, wherein: the first circadian rhythm index or the second circadian rhythm index is calculated by a first parameter, a second parameter, and a third parameter, and
the first parameter is a timing related to light irradiation, the second parameter is a weight at the specific timing, and the third parameter is the circadian illuminance value.

16. The operation method for analyzing a circadian rhythm index of claim 15, further comprising:
calculating the first circadian rhythm index or the second circadian rhythm index by Equation 1,
$$Cir = {\int }_{0}^{24} w ( t ) B ( t ) dt = {\int }_{0}^{24} w ( t ) G α ( t ) ( 1 - n ( t ) ) dt \qquad [ Equation l ]$$
where w(t) represents the second parameter, G represents a constant, α(t) represents a ratio at which a state of a light receptor is changed from ready to used, and (1−n(t)) represents a ratio of light receptors in the ready state among all light receptors.

17. The operation method for analyzing a circadian rhythm index of claim 16, further comprising:
calculating n(t) by Equation 2,
$$n ( t ) = ( n ( 0 ) - \frac{{α}_{f}}{{α}_{f} + β} ) {e}^{- ( {α}_{i} + β ) t} + \frac{{α}_{f}}{{α}_{f} + β} \qquad [ Equation 2 ]$$
where β represents a ratio at which the state of the light receptor is changed from used to ready, and α represents a constant value of α(t) when the circadian illuminance value is maintained constantly during a predetermined period.

18. The operation method for analyzing a circadian rhythm index of claim 17, further comprising:
calculating α(t) or n(t) by using the third parameter for each time which is a constant value when the predetermined period is 1 h; and
calculating the first circadian rhythm index or the second circadian rhythm index by Equations 3 and 4,
$$Cir = {\sum }_{h = 1}^{24} {w}_{h} {B}_{5} ( h ) \qquad [ Equation 3 ]$$
$${B}_{s} ( h ) = \frac{1}{G} {\int }_{h - 1}^{b} B ( t ) d t = {\int }_{h - 1}^{h} α ( t ) ( 1 - n ( t ) ) dt \qquad [ Equation 4 ]$$
where h represents a timing of a 1 h unit, and Wh represents an average value of the second parameter between (h−1) and h.

19. The operation method for analyzing a circadian rhythm index of claim 12, further comprising:
calculating a sleep disturbance index based on the second circadian rhythm index,
wherein the sleep disturbance index is an indicator indicating a quality of sleep determined by the external light.

20. The operation method for analyzing a circadian rhythm index of claim 19, wherein the first circadian rhythm index or the second circadian rhythm index is calculated by a first parameter, a second parameter, and a third parameter, and
the first parameter is the timing related to light irradiation, the second parameter is a light irradiation duration time, and the third parameter is the circadian illuminance value.

21. The operation method for analyzing a circadian rhythm index of claim 20, wherein the first circadian rhythm index is a circadian phase shift (CPS) value calculated based on an absolute timing corresponding to a predetermined dim light melatonin onset (DLMO).

22. The operation method for analyzing a circadian rhythm index of claim 21, wherein the second circadian rhythm index is a melatonin suppression response (MSR) value calculated based on an absolute timing corresponding to a predetermined dim light melatonin onset (DLMO).

23. The operation method for analyzing a circadian rhythm index of claim 22, further comprising:
outputting the light pollution index or the sleep disturbance index.

24. A computer program stored in a computer readable recording medium to perform the operation method for analyzing a circadian rhythm index according to claim 12 in combination with a computer which is hardware.
